(19) United States
(12) Patent Application Publication
(10) Pub. No.: US 2013/0237962 A1
(43) Pub. Date: Sep. 12, 2013

(54) CATHETER ASSEMBLY

(71) Applicant: Jun Kawai, Otsu-shi (JP)

(72) Inventor: Jun Kawai, Otsu-shi (JP)

(73) Assignee: GOODMAN CO., LTD., Nagoya-shi, Aichi (JP)

(21) Appl. No.: 13/521,992

(22) PCT Filed: Nov. 16, 2010
(86) PCT No.: PCT/JP2010/070396

§ 371 Date: May 21, 2013

(30) Foreign Application Priority Data
Jan. 14, 2010 (JP) 2010-006217

(51) Int. Cl. A61M 25/00

(52) U.S. Cl. CPC A61M 25/0054
USPC 604/524; 604/264

(57) ABSTRACT

Provided is a catheter assembly that can increase the degree of freedom for selecting a guide wire to be used. The catheter assembly includes an outer catheter and an inner catheter. The outer catheter is a catheter for introducing a balloon catheter or the like to an affected area and the inner catheter is an insertion-assisting tool that assists insertion by preceding the insertion of the outer catheter when being inserted in a human body. An inner opening by which a guide wire tube hole of the inner catheter is opened at a proximal end side and an outer opening by which the outer tube hole of the outer catheter is opened at a proximal end side are formed at the middle position in the axial line direction, and the openings are formed at an angle with respect to the axial line directions at positions on the same line.

### CROSS REFERENCE TO RELATED APPLICATION

This application claims the priority of Japanese Patent Application No. 2010-006217, filed on Jan. 14, 2010 in the JPO (Japan Patent Office). Further, this application is the National Phase application of International Application No. PCT/JP2010/070396 filed Nov. 16, 2010, which designates the United States and was published in Japanese.

### TECHNICAL FIELD

The present invention relates to a catheter assembly, the distal end side of which is introduced into an introduction target part such as the coronary arteries, when used.

### BACKGROUND ART

As a catheter assembly of which the distal end side is introduced into an introduction target part such as the coronary arteries in use, a catheter assembly having a tube-shaped outer member and a tube-shaped inner member at least partially inserted in a tube hole of the outer member has been known.

As the catheter assembly, for example, a configuration having a catheter as an outer member and an insertion assisting tool as an inner member has been disclosed in Patent Document 1. In this case, with the insertion assisting tool disposed such that a portion protrudes toward the distal end side from an opening at the front end of the catheter, the catheter assembly is introduced in a body and the insertion assisting tool is pulled out after being introduced. Further, a balloon catheter is newly inserted for the catheter left in a body and the affected area is repaired.

In the configuration of Patent Document 1, a guide wire is used when introducing a catheter assembly into a body, and the guide wire is inserted in a tube hole of the insertion assisting tool. In the configuration, the insertion assisting tool has a front end having a tube shape and a shaft. The shaft extends from the front end to a proximal end side and has an outer diameter smaller than that of the front end. Accordingly, it is possible to smoothly move the insertion assisting tool forward/backward in the catheter while decreasing the contact area between the inner circumferential surface of the catheter and the outer circumferential surface of the insertion assisting tool, in comparison to the configuration in which a tube hole for inserting a guide wire is formed throughout the axial line direction of the insertion assisting tool. Further, since the lumen of the front end is open to the proximal end side, it is possible to prevent the guide wire from locking to the catheter when pulling out or inserting the guide wire, with the insertion assisting tool inserted in the catheter, thereby making the operation easy.

Patent Document 1: Japanese Patent Application Laid-Open No. 2008-142351

However, in the configuration of Patent Document 1, when the catheter assembly is taken out from a body or replaced, with the front end of the guide wire left in the repair object part in the body, it is necessary to remove the entire catheter assembly from the body while keeping a portion of the guide wire at the proximal end side further than the catheter. And then, it is necessary to separate the catheter assembly while holding a portion of the guide wire that is exposed at the distal end side further than the catheter assembly. Therefore, the guide wire that is used is required to have a length equal to or more than the sum of the distance from the introduction start position to the repair object part in the body and the length of the catheter assembly. Thus it is necessary to use a guide wire having a large length.

### DISCLOSURE OF THE INVENTION

The present invention has been made in consideration of the situations and it is an object to provide a catheter assembly that can increase the degree of freedom in selection of a guide wire to use.

Hereinafter, means useful to solve the problems will be described, if necessary, together with the operation, effect, and the like.

A catheter assembly of the first aspect of the invention: includes: an inner member having an inner hole through which a guide wire is inserted; and an outer member having an outer hole through which the inner member is inserted. The inner member has an inner opening, by which the inner hole is opened to the outside of the inner member, at a middle position in an axial line direction of the inner member. The inner opening is formed such that an inner open surface faces a proximal end side or inclines with respect to the axial line direction, and the outer member has an outer opening, by which the outer hole is opened to the outside of the outer member, at a middle position in an axial line direction of the outer member. The outer opening is formed such that an outer open surface faces a proximal end side or inclines with respect to the axial line direction, on the same line as the inner opening.

According to this configuration, since the inner opening and the outer opening are formed at the middle position of the catheter assembly in the axial line direction, it is possible to use a guide wire with a small length. In this case, the outer opening is formed on the same line as the inner opening and the inner opening and the outer opening have a component facing the proximal end side as the component that the opening surfaces face, such that the guide wire is less bent in the direction crossing the axial line direction when being inserted or drawn out and the work of inserting or drawing out can be smoothly performed.

The catheter assembly of the second aspect of the invention: in the first aspect of the invention, the inner member has an inner tube having the inner hole and the inner opening at the proximal end portion, and an inner shaft being provided to extend toward the proximal end side from the inner tube and having a dimension that is smaller than a dimension of the inner opening in a direction perpendicular to the axial line direction. The outer member has an outer tube having the outer hole and the outer opening at the proximal end portion, and an outer shaft being provided to extend toward the proximal end side from the outer tube and having a dimension that is smaller than a dimension of the outer opening in a direction perpendicular to the axial line direction.

According to this configuration, when the guide wire passes through the catheter assembly, the portion of the proximal end side further than the inner opening of the inner member or the portion of the proximal end side further than the outer opening of the outer member does not become an obstacle.

The catheter assembly of the third aspect of the invention: in the second aspect of the invention, includes: a contrast being provided at a position of the opening of the one of the inner member and the outer member, or a position adjacent thereto; and a joint part being provided at the other one of the inner member and the outer member and having a metal region welded to a metal region of the shaft of the other one of the inner member and the outer member, and moreover connecting the shaft with the corresponding tube by welding. The joint part is provided not to overlap the contrast portion in the axial line direction.

According to this configuration, it is possible to strongly connect the corresponding tube with the shaft, at the side where the joint part is disposed, in at least one with the joint part in the inner member and the outer member. In this case, since the joint part is disposed not to overlap the contrast portion, it is possible to perform the strong connection while showing the function of the contrast portion well.

The catheter assembly of the fourth aspect of the invention: in the second aspect of the invention, the outer tube has a joint part being provided at a position of the outer opening or a position adjacent thereto, and having a metal region welded to a metal region of the outer shaft, and moreover connecting the outer shaft to the outer tube by welding. According to this configuration, since it is possible to strongly connect the outer tube with the outer shaft and to contrast the joint part, it is possible to recognize the position of the outer opening, with the catheter assembly inserted in a body, through the contrasting.

The catheter assembly of the fifth aspect of the invention: in the fourth aspect of the invention, the inner opening is positioned further toward a distal end side than the outer opening when relative positions of the inner member and the outer member in the axial line direction are predetermined initial positions. The inner member has a contrast portion provided at the position of the inner opening or a position adjacent thereto, and the length of the joint part is set not to overlap the contrast portion in the axial line direction. According to this configuration, it is possible to show the function of the joint part without reducing the function of the contrast portion for the inner opening while using the joint part as a marker for contrasting the outer opening.

The catheter assembly of the sixth aspect of the invention: in the fourth or fifth aspect of the invention, the inner opening is positioned further toward a distal end side than the outer opening when relative positions of the inner member and the outer member in the axial line direction are predetermined initial positions. And the inner shaft has a rigidity reduction structure at the position overlapping the joint part in the axial line direction when the relative positions are the initial positions. The rigidity reduction structure exhibits reduced rigidity of the inner shaft than that on the proximal end side. According to this configuration, in the configuration where the joint part made of metal is disposed for the outer member, it is possible to suppress a local increase in rigidity by using the inner shaft.

The catheter assembly of the seventh aspect of the invention: in any one of the fourth to sixth aspects of the inventions, the joint part is formed to have a cylindrical shape around the axial line and an open surface of a proximal end side thereof inclines with respect to the axial line. The outer shaft has a tapered portion at a distal end portion thereof, the tapered portion is formed by crushing one side of the outer shaft to the other side with the axial line therebetween, and has a large width surface and an inclining surface. The large width surface includes a portion leveled with the portion positioned further toward the proximal end side than the tapered portion throughout the axial line direction and is made wider than the portion on the proximal end side. And the inclining surface is inclined to gradually decrease the distance from the large width surface toward the distal end side. The axial line direction of the outer shaft is the axial line direction of the outer tube as the large width surface is provided to be in contact from the inside with a portion, the portion being the inner circumferential surface of a tube wall defining the opening and the periphery of the proximal end side of the joint part and being in the same straight line of the proximal end portion of the opening. The joint part and the outer shaft are welded at the contact portion.

According to this configuration, as the large width surface is in contact from the inside with the inner circumferential surface of the joint part and welded, it is possible to increase the strength of connection while suppressing a step at the outer circumference of the joint part. Further, since the large width surface has a portion leveled with the proximal end side further than that throughout the axial line direction, it is possible to arrange the axial line direction of the outer shaft in the axial line direction of the outer tube, when the large width surface is in contact with the inner circumferential surface of the joint part. Further, in the configuration in which the opening of the proximal end side of the joint part is formed to be inclined, since the large width surface is in contact with the portion in the same straight line as the proximal end portion at the opening of the proximal end side and the inclining surface of the tapered portion faces the inside, it is possible to show excellent effect as described above while suppressing a decrease in passing performance of the guide wire at the outer opening.

### BRIEF DESCRIPTION OF THE DRAWINGS

FIG. 1(a) is a front view showing the configuration of an outer catheter and FIG. 1(b) is a front view showing the configuration of an inner catheter.

FIG. 2 is a vertical cross-sectional view of a distal end side of the inner catheter.

FIG. 3(a) is a partial front view enlarging an outer tube and the peripheral, FIG. 3(b) is a vertical cross-sectional view enlarging the periphery of the joint of the outer tube and an outer shaft, FIG. 3(c) is a front view showing a joint ring and the distal end side of the outer shaft, and FIG. 3(d) is a view enlarging the joint of the joint ring and the outer shaft.

FIG. 4(a) is a front view of a catheter assembly when being inserted in a guiding catheter, with a guide wire inserted and FIG. 4(b) is a cross-sectional view taken along line A-A.

FIG. 5 is an illustrative view for illustrating the shape when a catheter assembly is introduced in a left coronary artery.

FIG. 6 is a partial front view for illustrating another shape of the outer catheter.

### BEST MODE FOR CARRYING OUT THE INVENTION

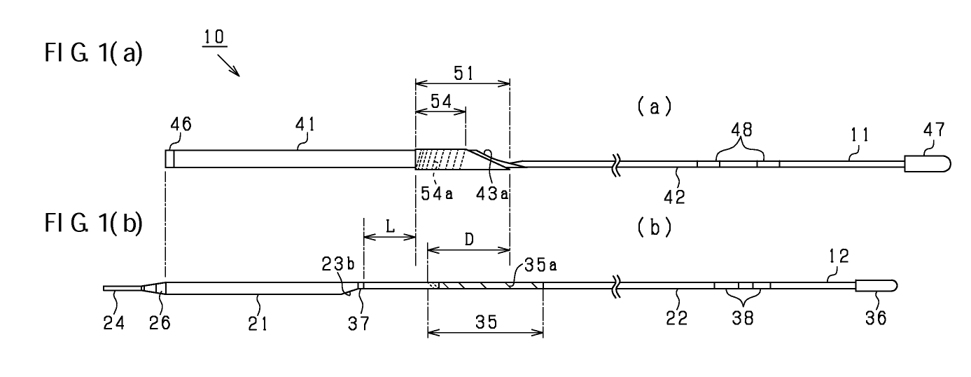

Hereinafter, an embodiment when the present invention is applied to a catheter assembly will be described with reference to the drawings. FIGS. 1(a) and 1(b) are front views showing members constituting a catheter assembly 10.

As shown in FIGS. 1(a) and 1(b), the catheter assembly 10 includes an outer catheter 11 disposed as an outer member and an inner catheter 12 disposed as an inner member. The outer catheter 11 is a catheter for delivering that is used to introduce a balloon catheter or the like which is not shown, into a peripheral stenosis affected area of a coronary artery. The inner catheter 12 is inserted and used in the outer catheter 11 when the outer catheter 11 is inserted in an affected area in a body. The inner catheter 12 is an insertion assisting tool that assists the insertion positioned ahead of the outer catheter 11. The entire lengths of both the outer catheter 11 and the inner catheter 12 are 1500 mm, but may be larger or smaller.

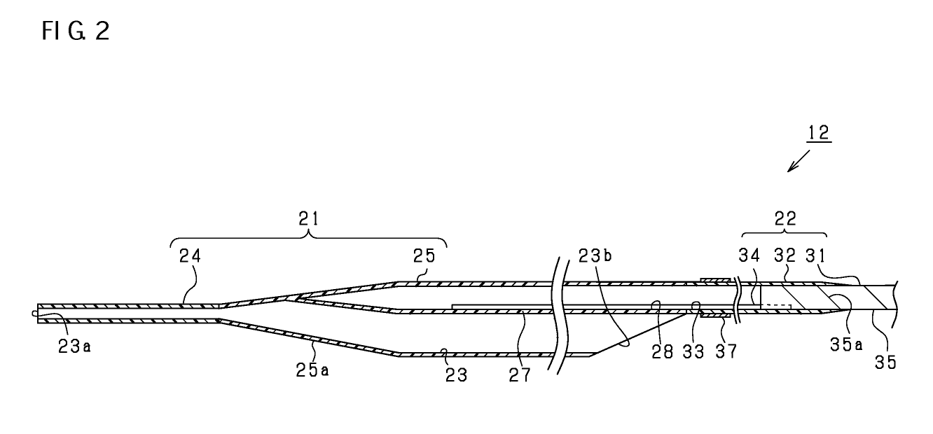

The inner catheter 12 in both catheters 11 and 12 is described first with reference to FIG. 1(b) and FIG. 2. FIG. 2 is a vertical cross-sectional view of the distal end side (front end side) of the inner catheter 12.

The inner catheter 12, as shown in FIG. 1(b), has an inner tube 21 that is the part from the distal end to the middle position of the proximal end side (base end side) and constitutes the distal end side of the inner catheter 12 and an inner shaft 22 constituting the proximal end side further than the inner tube 21. The inner tube 21, as shown in FIG. 2, has a tube hole (lumen) 23 formed throughout the axial line direction by opening both the distal end portion and the proximal end portion, as an inner hole, and generally has a tube shape. The tube hole 23 is used to insert a guide wire. Hereinafter, the tube hole 23 is also referred to as a guide wire tube hole (guide wire lumen) 23.

The inner tube 21 is formed such that the outer diameter and the diameter of the hole of the guide wire tube hole 23 gradually decrease toward the distal end side from the middle position in the axial line direction. The distal end portion of the inner tube 21 is configured by a soft tip 24 having constant outer diameter and inner diameter throughout the axial line direction. That is, the inner tube 21 has a base tube 25 and the soft tip 24 constituting the distal end side further than the base tube 25. The base tube 25 has a tapered region 25a of which the diameter gradually decreases toward the soft tip 24 from the middle position in the axial line direction. Further, the guide wire tube hole 23 is formed by the tube holes of the base tube 25 and the soft tip 24 that communicate with each other, a proximal end opening 23a of the tube hole 23 is formed by the distal end portion of the soft tip 24, and a proximal end opening 23b of the tube hole 23 is formed by the proximal end portion of the base tube 25.

Both the base tube 25 and the soft tip 24 are made of PEBAX (Polyether Block amide) copolymer, but the soft tip 24 is formed to be more flexible than the base tube 25. The base tube 25 and the soft tip 24 are thermally welded. Further, a marker 26 for a tapered region that is made of a material such as tungsten that blocks radiation (X-rays) and is formed in a cylindrical shape, as shown in FIG. 1(b), is disposed at the end of the proximal end side of the tapered region 25a of the base tube 25.

The inner shaft 22 fixed to the inner tube 21, as shown in FIG. 2, has a hypotube 31 formed in a tube shape. The inner shaft 22 also has an extending tube 32 covering the outer circumference of the distal end portion of the hypotube 31 and extending the inner shaft 22 to the distal end side further than the hypotube 31. The hypotube 31 is made of metal such as stainless steel or a Ni—Ti alloy in a tube shape and the extending tube 32 is made of synthetic resin such as polyamide in a tube shape. The members 31 and 32 are connected by the extending tube 32 thermally welded to the hypotube 31. Further, the outer circumferential surface of the hypotube 31 may be coated with synthetic resin. In the hypotube 31 and the extending tube 32, the extending tube 32 is thermally welded to the base tube 25, such that the inner shaft 22 is connected to the inner tube 21.

Describing the configuration according to the connection in detail, a connection tube 27 having the same outer diameter and inner diameter as those of the extending tube 32 is formed from the proximal end opening 23b of the guide wire tube hole 23 to the position of the tapered region 25a, in the base tube 25. A connection tube hole (connecting lumen) 28 extending in the axial line direction is formed at the connection tube 27. The distal end side of the connection tube hole 28 is closed by a wall constituting the tapered region 25a but the proximal end side is open. The extending tube 32 is thermally welded to the connection tube 27 such that a shaft-side tube hole (shaft-side lumen) 33 that is configured by the tube holes (lumens) of the hypotube 31 and the extending tube 32 communicates with the connection tube hole 28.

A core material 34 having an outer diameter smaller than those of the tube holes 28 and 33 is disposed to cross the interfaces of both the tube holes 28 and 33 by using communication of the connection tube hole 28 and the shaft-side tube hole 33. The core material 34 is a wire made of metal such as stainless steel or a Ni—Ti alloy and bonded to the inner circumferential surface of the hypotube 31. Further, the end of the distal end side reaches even the middle position of the base tube 25 in the axial line direction. As the core material 34 is disposed, kink resistance of the connection portion of the inner tube 21 and the inner shaft 22 is improved.

Note that the core material 34 is not fixed with respect to the connection tube hole 28 or the extending tube 32, but may be fixed. In this case, the core material 34 is integrated well to the connection tube 27 and the extending tube 32. As a more preferable fixing configuration, a configuration of filling the connection tube hole 28 or the tube hole of the extending tube 32 with synthetic resin, with the core material 34 inserted, or a configuration of making the outer diameter of the core material 34 and the diameters of the connection tube hole 28 and the tube hole of the extending tube 32 substantially the same to forcibly press the core material 34 into the tube holes, is considered.

Further, as the core material 34 is disposed to be inserted in the inner tube 21 from the hypotube 31, a local change in rigidity at the position of the distal end portion of the hypotube 31 is suppressed and the kink resistance is also improved. In addition, as means for suppressing a local change in rigidity at the distal end portion of the hypotube 31, an inner side rigidity reduction structure is disposed for the hypotube 31, other than the core material 34.

Describing the inner side rigidity reduction structure in detail, as shown in FIG. 1(b) and FIG. 2, an inner side rigidity reduction region 35 is formed by a spiral slit 35a from the middle position in the axial line direction to the distal end portion, in the hypotube 31. The slit 35a is formed such that the pitch between slits adjacent to each other in the axial line direction decreases at the distal end side relatively to the proximal end side. That is, the inner side rigidity reduction region 35 is formed such that the rigidity of the hypotube 31 gradually decreases toward the distal end side. Further, as shown in FIG. 2, the slit 35a is also formed at the position covered by the extending tube 32, in the hypotube 31.

The inner shaft 22 in the above configuration has a substantially constant outer diameter throughout the axial line direction, except for an inner grip 36 disposed at the end of the proximal end side and the outer diameter is substantially the same as the outer diameter of the connection tube 27, as described above.

Since the proximal end opening 23b of the guide wire tube hole 23 is formed at the proximal end portion of the inner tube 21, the position is the middle position of the inner catheter 12 in the axial line direction. That is, an inner port for drawing out the guide wire at the proximal end side is formed at the middle position of the inner catheter 12 in the axial line direction. Further, in the following description, the proximal end opening 23b is also referred to as an inner opening 23b for the convenience of description.

The inner opening 23b is formed with an inner open surface at an angle with respect to the axial line direction, and a wide open area is ensured. Further, the inner shaft 22 is disposed to be biased at the portion that is the most proximal end side in the inclined inner opening 23b. The portion that is the most distal end side in the inner opening 23b is opposite to the inner shaft 22 with an axial line therebetween. Therefore, the inner opening 23b has a directional component facing the proximal end side and to release the guide wire tube hole 23 to a side. Further, the inner opening 23b has a dimension in the direction perpendicular to the axial line direction that is larger than the dimension of the inner shaft 22 and the difference is equal to or more than the outer diameter of the guide wire to be used.

A marker 37 for an inner opening is disposed as a contrast portion, as shown in FIG. 1(b) and FIG. 2, at a position close to or adjacent to the proximal end side with respect to the inner opening 23b in the inner shaft 22. The marker 37 is made of a material such as tungsten blocking radiation in a cylindrical shape. The position where the marker 37 is disposed is a position spaced at 230 mm to the proximal end side with respect to the distal end portion of the inner catheter 12. The position is spaced to the distal end side further than the distal end portion of the hypotube 31. As the marker 37 for an inner opening is disposed, it is possible for an operator to know the position of the inner opening 23b even though the catheter assembly 10 is inserted in a body.

Note that the material of the marker 37 for an inner opening is not limited to tungsten and may be gold, platinum, iridium, barium, barium sulfate, bismuth, bismuth oxide, bismuth oxycarbonate, bismuth subcarbonate, zirconium oxide, tantalum, a cobalt chrome alloy, sodium iodide, silver-protein colloid, silver iodide-gelatin colloid, stainless steel, titanium or the like. Further, the marker 37 for an inner opening may be disposed at the periphery of the inner opening 23b. A marker 38 for visually checking the insertion amount of the inner catheter 12 in a body is also disposed at the proximal end side of the inner shaft 22, as shown in FIG. 1(b).

Next, the outer catheter 11 is described.

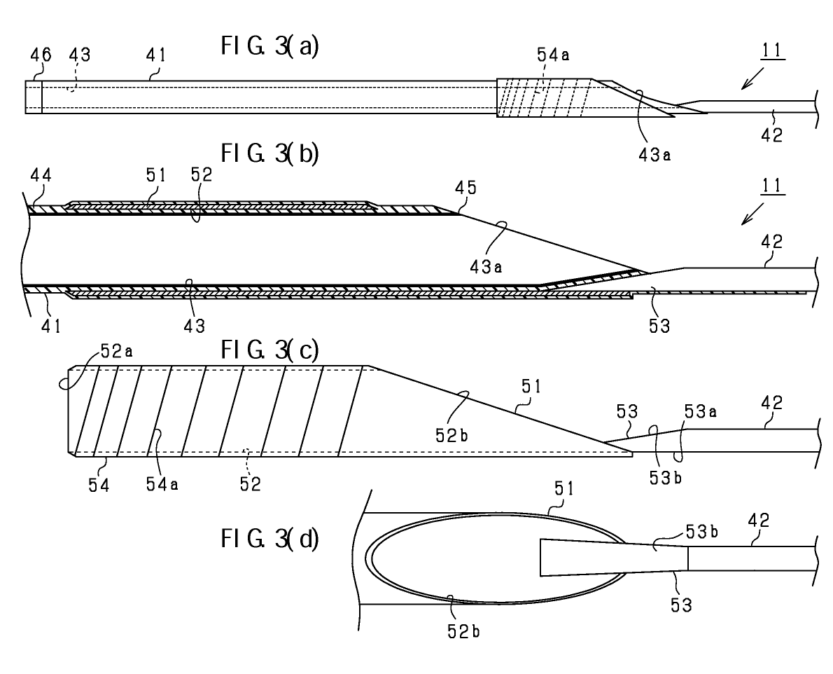

The outer catheter 11, as shown in FIG. 1(a), has an outer tube 41 constituting the distal end side of the outer catheter 11 from the distal end to the middle position of the proximal end side (base end side) and an outer shaft 42 constituting the proximal end side further than the outer tube 41. The detailed configuration of the outer tube 41 and the outer shaft 42 are shown in FIG. 3.

FIG. 3(a) is a partial front view enlarging the outer tube 41 and the periphery, FIG. 3(b) is a vertical cross-sectional view enlarging the connection portion between the outer tube 41 and the outer shaft 42 and the periphery, FIG. 3(c) is a front view showing a joint ring 51 and the distal end side of the outer shaft 42, and FIG. 3(d) is a view enlarging the connection portion between the joint ring 51 and the outer shaft 42.

The outer tube 41, as shown in FIG. 3(a) and FIG. 3(b), has an outer tube hole (outer lumen) 43 formed throughout the axial line direction by opening both the distal end portion and the proximal end portion, as an outer hole, and generally has a tube shape. The outer tube hole 43 is used to insert the inner catheter 12 and a guide wire, and a catheter such as a balloon catheter that is used for an affected area is inserted, when the inner catheter 12 is separated.

The outer tube hole 43 has a constant hole diameter substantially throughout the axial line direction. The hole diameter of the outer tube hole 43 is slightly larger than the outer diameter of the region between the tapered region 25a and the inner opening 23b in the inner tube 21, that is, the maximum outer diameter of the inner tube 21. In the outer tube 41, a base layer 44 including the outer circumferential surface is made of PEBAX, such that flexibility that allows following a curved blood vessel is ensured. And a friction reduction layer 45 made of Teflon (registered trademark) is formed on the inner circumferential surface of the outer tube 41 to reduce resistance when the inner catheter 12 or other catheters slides in the outer tube hole 43. The friction reduction layer 45 is disposed to cover the entire inner circumferential surface or may be partially disposed in a portion where sliding is easy to occur but not in a portion where sliding is difficult to occur.

The material of the base layer 44 is not limited to a polyamide elastomer such as PEBAX, and polyethylene, polyethylene terephthalate, polypropylene, polyurethane, polyimide, polyimide elastomer, silicone rubber, natural rubber, or the like may be used. Further, the materials may be used for the inner tube 21. In addition, the material of the friction reduction layer 45 is not limited to Teflon, and other fluorine-based resin or hydrophilic polymer such as maleic anhydride copolymer may be used.

The outer tube 41 has predetermined flexibility, as described above, but has an outer side tip 46 having higher flexibility at the distal end portion than the proximal end side. Accordingly, even if the distal end portion of the outer tube 41 comes in contact with the wall of a blood vessel, load applied to the wall of the blood vessel decreases. The outer side tip 46 is made of the same material as that of the base layer 44, but may be made of other materials.

The outer tube 41 has a length of 300 mm that is larger than the length of the inner tube 21. Therefore, when the inner tube 21 is inserted in the outer tube 41 such that the soft tip 24 and the tapered region 25a are positioned at the distal end side further than the outer tube 41, the proximal end portion of the inner tube 21 is positioned at the distal end side further than the proximal end portion of the outer tube 41. The relative positions are described in detail below.

The outer shaft 42 fixed to the outer tube 41 is made of metal such as stainless steel or a Ni—Ti alloy in a cylindrical shape. The outer shaft 42, as shown FIG. 3(b) and FIG. 3(c), is connected to the outer tube 41 by the joint ring 51 disposed as a joint part. Further, the outer circumferential surface of the outer shaft 42 may be coated with synthetic resin within a range not interfering with the connection.

The configuration according to the connection is described in detail. The joint ring 51 is made of metal such as stainless steel in a cylindrical or tube shape and a joint tube hole (joint lumen) 52 is open at both ends in the axial line direction. In this case, as shown in FIG. 3(c), an opening 52a is formed such that the open surface is perpendicular to the axial line of the joint ring 51, while the other opening 52b is formed such that the open surface inclines with respect to the axial line of the joint ring 51. Further, the joint ring 51 has constant outer diameter and inner diameter, except for the place where the other opening 52b is formed. Note that in the following description, the other opening 52b is also referred to as a joint side-inclining opening 52b.

The joint ring 51 is disposed at the outer tube 41 such that the joint side-inclining opening 52b becomes the proximal end side and the outer shaft 42 is jointed with respect to the opening 52b. The outer shaft 42 deforms such that the end of the distal end portion is tapered.

The tapered portion 53, as shown in FIG. 3(c) and FIG. 3(d), is not formed such that the entire circumferential surface is tapered toward the front end, but formed to be tapered by crushing one side to the other side with the axial line therebetween. In this case, the tapered portion 53 has a large width surface 53a and an inclining surface 53b. The large width surface 53a includes a portion leveled with the portion positioned further toward the proximal end side than the tapered position 53 throughout the axial line direction and is made wider than the portion on the proximal end side. The inclining surface 53b is inclined to gradually decrease the distance from the large width surface 53a toward the distal end side.

The tapered portion 53 is disposed such that the large width surface 53a is in contact from the inside with the inner circumferential surface of the peripheral portion inclining to the proximal end side of the join side-inclining opening 52b, and the inner circumferential surface and the large width surface 53a are bonded. The bonding is performed by radiating a laser to a plurality of positions (in detail, two positions) to generate welded positions at a plurality of positions spaced in the axial line direction. Thus, the joint ring 51 and the outer shaft 42 are strongly fixed. Note that in the large width surface 53a, the curvature around the axis and the curvature of the inner circumferential surface of the contact portion are substantially constant.

Describing the bonding positions in more detail, the most front end of the tapered portion 53 is positioned at a proximal end side further than the periphery that is the most distal end side of the joint side-inclining opening 52band not to be covered by the periphery. Accordingly, it is easy to radiate a laser and to suppress the area of the region where the joint ring 51 and the outer shaft 42 overlap each other in the axial direction, thereby reducing influence on the rigidity.

However, as the overlap region between the joint ring 51 and the outer shaft 42 is narrowed in the configuration, they may not be strongly fixed, but with the large width surface 53a of the tapered portion 53 in contact with the periphery of the joint side-inclining opening 52b, it is possible to widen the range of the welded position, as compared with when the large width surface 53a is not generated. Therefore, sufficient bonding strength is achieved. Further, as the large width surface 53a is bonded while being in contact, the axial line direction of the outer shaft 42 is parallel with the axial line direction of the joint ring 51 (that is, the axial line direction of the outer catheter 11).

When further increasing the bonding strength, it is considered to integrally form the inclining portion, which is the most proximal end side of the periphery of the joint side-inclining opening 52b, with the extending portion that further extends to the proximal end side from it.

The joint ring 51 connected with the outer shaft 42, as shown in FIG. 3(b), is embedded in a base layer 44 of the outer tube 41. That is, the joint ring 51 has a length set to be smaller than the length of the base layer 44 and the joint ring 51 is positioned at the middle position of the outer tube 41 in the axial line direction.

In the configuration with the joint ring 51 embedded in the base layer 44, a local increase in rigidity may be generated at the position where the joint ring 51 is disposed. Accordingly, an outer side rigidity reduction structure is disposed at the joint ring 51.

For the outer side rigidity reduction structure, in detail, as shown in FIG. 3(c), an outer rigidity reduction region 54 is formed by a spiral slit 54a, from the distal end side of the joint ring 51 in the axial line direction to the portion positioned ahead of the joint side-inclining opening 52b. The slit 54a is formed such that the pitch between the slits adjacent to each other in the axial line direction is small at the distal end side relative to the proximal end side. That is, the outer side rigidity reduction region 54 is formed such that the rigidity of the joint ring 51 gradually decreases toward the distal end side. Therefore, it is possible to suppress a local increase in rigidity and improve kink resistance.

A method of embedding a joint ring 51 into a base layer 44 is described.

First, as a preparation process, the outer shaft 42 is bonded with respect to the joint ring 51. Further, an outer layer tube made of PEBAX as a single layer tube and an inner layer tube made of PEBAX and Teflon as an inner-and-outer double layer tube are prepared. Note that the lengths of the outer layer tube and the inner layer tube are the same.

Thereafter, a process of disposing the outer layer tube and the inner layer tube to fit the joint ring 51 between the layers in the radial direction and a process of thermally welding the inner and outer overlap portions of the outer layer tube and the inner layer tube are performed. The thermal welding is performed by using a shaft for thermal welding or the like such that the inner diameter of the outer tube hole 43 becomes constant in the axial line direction, except for the proximal end side where the tapered portion 53 of the outer shaft 42 exists. As the inner diameter becomes constant, a step is formed on the outer circumferential surface of the outer tube 41, but the thickness of the base layer 44 may be set such that the step is not generated.

As the thermal welding is performed, as described above, the joint ring 51 is fitted by the base layer 44 from the distal end side and the proximal end side, such that the joint ring 51 is prevented from displacing from the position in the axial line direction. Further, in the thermal welding, the outer layer tube is heated from the outer circumferential surface, and the heating is not performed on the outer side rigidity reduction region 54 of the joint ring 51. Therefore, it is possible to show well the function of the outer side rigidity reduction region 54. Meanwhile, heating is performed on the region where the outer side rigidity reduction region 54 is not disposed and the region where the tapered portion 53 of the outer shaft 42 is disposed. Therefore, the portions where the base layer 44 is thermally welded exist in the joint ring 51 or the outer shaft 42, such that the connection can be strongly performed.

The joint side-inclining opening 52b is formed at the joint ring 51, as described above, and a proximal end opening 43a of the outer tube hole 43 exists at a position further toward the proximal end side than the position of the inclining opening 52b at the outer tube 41. Since the proximal end opening 43a is formed at the proximal end side of the outer tube 41, the position is the middle position of the outer catheter 11 in the axial line direction. A guide wire that is used when the catheter assembly 10 is inserted into a body passes through the outer tube hole 43 at a position further toward the proximal end side than the inner opening 23b, in addition to passing the guide wire tube hole 23 of the inner catheter 12. In this case, as the proximal end opening 43a of the outer tube hole 43 is formed at the position, it can be stated that the outer port for drawing out the guide wire passing through the outer tube hole 43 out of the outer catheter 11 at the proximal end side is formed at the middle position of the outer catheter 11 in the axial line direction. Note that in the following description, the proximal end opening 43a is also referred to as an outer opening 43a for the convenience of description.

The outer opening 43a is formed with an outer open surface at an angle with respect to the axial line direction, and a wide open area is ensured. Further, the portion that is the most distal end side of the outer opening 43a is opposite the outer shaft 42 with the axial line therebetween. Therefore, the outer opening 43a has a component facing the proximal end side and is shaped to release the outer tube hole 43 to a side.

The outer opening 43a has a dimension in the direction perpendicular to the axial line direction that is larger than a dimension of the outer shaft 42 and the difference is equal to or more than the outer diameter of the guide wire to be used. Further, an inclining surface 53b is formed at the opposite side of the large width surface 53a in the tapered portion 53 of the outer shaft 42. The inclining surface 53b goes to the outer circumference as the inclining surface 53b goes to the distal end side. Accordingly, the operability of the guide wire is improved.

The joint ring 51 made of stainless steel is disposed at the position adjacent to the outer opening 43a, as described above. Since stainless steel blocks radiation, when radiation is irradiated with the catheter assembly 10 inserted in a body, the joint ring 51 is contrasted, such that it is possible to know the position of the outer opening 43a. That is, the joint ring 51 has the function of a marker for showing the position of the outer opening 43a. Further, since the joint ring 51 has strength higher than that of the base layer 44 in the radial direction, the outer opening 43a is not easily crushed. That is, the joint ring 51 has the function of a shape-holding member for making the outer opening 43a difficult to be crushed.

Further, the outer shaft 42, as shown in FIG. 1(a), has the outer diameter that is substantially constant throughout the axial line direction, except for the tapered portion 53 disposed at the end of the distal end side and the outer grip 47 disposed at the end of the proximal end side. In detail, it is 0.5 mm and this is the same or substantially the same as the outer diameter of the hypotube 31 of the inner shaft 22. In addition, two markers 48 for visually checking the amount of insertion of the outer catheter 11 in a body are disposed at the proximal end side of the outer shaft 42. One of the markers 48 is formed at the position of the proximal end side at 1000 mm from the distal end portion of the outer catheter 11. The position corresponds to the position where the outer catheter 11 starts to come out from a guiding catheter that is described below. Further, the other marker 48 is formed at the position of the proximal end side at 1200 mm from the distal end portion of the outer catheter 11. The position is for checking that another catheter such as a balloon catheter reaches the position of the outer opening 43a.

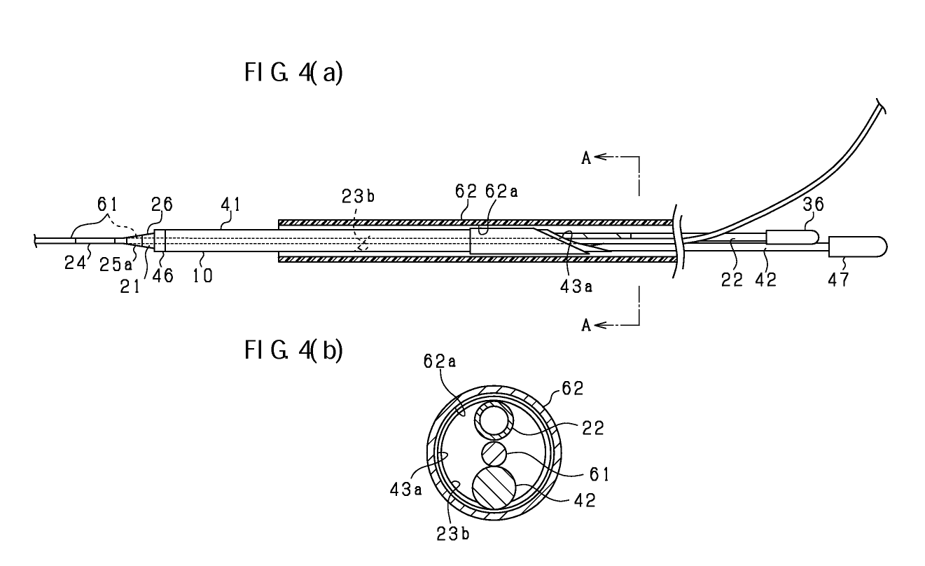

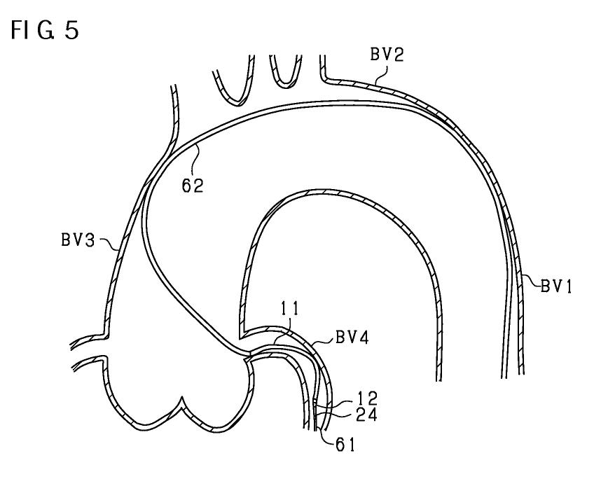

Next, when the catheter assembly 10 is formed by inserting the inner catheter 12 with respect to the outer catheter 11 is described with reference to FIG. 4 and FIG. 5.

FIG. 4(a) is a front view of the catheter assembly 10 when a guide wire 61 is inserted into the catheter assembly 10 and the catheter assembly 10 is inserted in the guiding catheter 62 and a vertical cross-sectional view of the guiding catheter 62 is shown. Further, FIG. 4(b) is a cross-sectional view taken along line A-A in FIG. 4(a). In addition, FIG. 5 is an illustrative view for illustrating the shape when the catheter assembly 10 is introduced in a left coronary artery BV4.

As shown in FIG. 4(a), in the initial state of the catheter assembly 10, the soft tip 24 and the tapered region 25a of the inner catheter 12 that is inserted in the outer tube 41 protrude to the distal end side further than the outer tube 41. The interface or the periphery of the tapered region 25a of the inner tube 21 and the region of the more proximal end side is positioned at the opening portion of the distal end side of the outer tube 41. This is the initial state of the catheter assembly 10.

As the inner catheter 12 goes before the outer catheter 11, as shown in FIG. 5, the distal end portion of the guiding catheter 62 is disposed at the inlet of the left coronary artery BV4 through a descending aorta BV1, an aortic arch BV2, and an ascending aorta BV3. When the catheter assembly 10 is protruded toward the distal end side from the state and inserted in the left coronary artery BV4, it is possible to allow the soft tip 24 first to follow the curved blood vessel and the passing performance can be increased. Further, when there is an occlusion portion, it is possible to slowly widen the occlusion portion from the soft tip 24 side. Note that the operation of making the outer catheter 11 go before the guiding catheter 62 is performed within a range where the outer opening 43a does not deviate from the guiding catheter 62.

Returning to the description of FIG. 4, in the initial state of the catheter assembly 10, the outer opening 43a of the outer catheter 11 is disposed at a position further toward the proximal end side than the inner opening 23b of the inner catheter 12. The position of the outer opening 43a is the middle position of the catheter assembly 10 in the axial line direction. In more detail, the position of the outer opening 43 is a position further toward a distal end side than the middle position of the entire length of the catheter assembly 10, and further toward a distal end side than a quarter from the distal end side of the catheter assembly 10. As the position of the outer opening 43a is set, as described above, when the catheter assembly 10 is introduced after the guide wire 61 goes first, or when the catheter assembly 10 or the outer catheter 11 is replaced after the catheter assembly 10 is introduced, it is possible to suppress the length of the guide wire 61, which is required to hold the guide wire 61 with hands at both sides in the axial line direction with the outer tube 41 therebetween, to be small. As a result, even the guide wire 61 of which the entire length is small can be used.

In the configuration in which the relative positions of the inner opening 23b and the outer opening 43a are the positions described above in the initial state, the regions that the guide wire 61 passes through are the guiding tube hole (guiding lumen) 62a formed at the guiding catheter 62→outer tube hole 43→guide wire tube hole 23, when seen from the proximal end side. That is, when the guide wire 61 is inserted from the proximal end side, the transverse area of the passed regions gradually decreases. Accordingly, it is possible to easily introduce the guide wire 61 to the distal end side.

However, when the positional relationship of the openings 23b and 43a is set, as described above, the outer shaft 42 and the inner shaft 22 are parallel with each other at the proximal end portion of the outer opening 43a and the left space in the guiding tube hole 62a is narrowed at the proximal end portion of the outer opening 43a. For this configuration, as shown in FIG. 4(b), the outer diameters of the shafts 22 and 42 are set such that the left space in the guiding tube hole 62a includes a space sufficiently larger than the transverse cross-section of the guide wire 61. Therefore, it is possible to smoothly draw out the guide wire 61 out of the outer opening 43a to the proximal end side or insert the guide wire 61 into the outer hole 43 through the outer opening 43a from the proximal end side.

Further, the outer opening 43a is disposed on the same line as the inner opening 23b, the open surfaces of the openings 23b and 43a are inclined, and the directional components facing the proximal end side are included in the directional components that the open surfaces face. Therefore, when the guide wire 61 introduced in the outer tube hole 43 from the outer opening 43a further goes, the front end of the guide wire 61 becomes easily introduced into the inner opening 23b. Similarly, the guide wire 61 forcibly pushed out of the inner opening 23b to the outer tube hole 43 becomes easily drawn out of the outer catheter 11 through the outer opening 43a.

Note that, when the catheter assembly 10 is introduced toward an affected area, the catheter assembly 10 may be forcibly inserted in the natural state or only the inner catheter 12 may be inserted into the affected area first within a range in which the inner opening 23b does not deviate from the outer catheter 11 to the distal end side and then the outer catheter 11 may be forcibly inserted. In the latter case, the relative positions of the inner opening 23b and the outer opening 43a are different from the natural state, but the inner opening 23b keeps positioned further toward the distal end side than the outer opening 43a.

Next, the positional relationship between the marker 37 for an inner opening and the joint ring 51 is described with reference to FIG. 1.

As described above, in the natural state of the catheter assembly 10, the inner opening 23b is disposed at a position further toward the distal end side than the outer opening 43a. In this case, the marker 37 for an inner opening that can show the position of the inner opening 23b under radiation is spaced by a distance L further toward the distal end side than the joint ring 51 that can show the position of the outer opening 43a. That is, in the configuration in which the joint ring 51 is disposed to be able to show the position of the outer opening 43a, the length of the joint ring 51 is set not to overlap the marker 37 for an inner opening. Accordingly, the marker 37 for an inner opening is individually contrasted, and in the configuration where the joint ring 51 is disposed, it is possible to clearly know the position of the inner opening 23b.

In particular, in the work of introducing the catheter assembly 10, the relative positions of the inner catheter 12 and the outer catheter 11 in the axial line direction may be changed. As described above, the change in relative position is generated when the inner catheter 12 goes first to a position further toward the distal end side than the position in the natural state. Therefore, in the change in relative position, the marker 37 for an inner opening is disposed to be further spaced from the joint ring 51 and it is possible to still contrast the marker 37 well.

Next, similarly, the positional relationship between the inner side rigidity reduction region 35 and the joint ring 51 is described with reference to FIG. 1.

In the natural state of the catheter assembly 10, the inner shaft 22 passes the position where the joint ring 51 of the outer tube 41 is disposed. Here, the inner side rigidity reduction region 35 exists in the passed region, as described above, and an overlap region D of the joint ring 51 and the inner side rigidity reduction region 35 exists. Accordingly, the rigidity locally increases at the position of the joint ring 51 in the outer catheter 11, the rigidity decreases at the position of the inner catheter 12 and the influence of an increase in rigidity due to the joint ring 51 can be suppressed in the catheter assembly 10.

Note that the distal end portion of the rigidity reduction region 35 is positioned further toward the proximal end side than the distal end portion of the joint ring 51, but it is not limited thereto and may be positioned at the same position in the axial line direction or maybe positioned at the distal end side.

According to the present embodiment described above, the following excellent effects are shown.

As the inner opening 23b and the outer opening 43a are formed at the middle position of the catheter assembly 10 in the axial line direction, it is possible to use the guide wire 61 of which the entire length is small. In this case, the inner opening 23b is formed at an angle to have a directional component facing the proximal end side in the direction that the open surface faces. The outer opening 43a is formed on the same line as the inner opening 23b, and is formed at an angle to have a directional component facing the proximal end side in the direction that the open surface faces. Accordingly, it is possible to easily draw out or insert the guide wire 61.

Since the outer tube 41 and the outer shaft 42 are connected by using the joint ring 51, it is possible to increase the connection strength well. In particular, in the catheter assembly 10, the inner catheter 12 is introduced with the outer catheter 11, but the outer catheter 11 is used to introduce another catheter while being independently held. Therefore, it is considered that large load is applied to the connection portion of the tube 41 and the shaft 42, at the outer catheter 11 in comparison to the inner catheter 12. Accordingly, resistance against the load is increased by using the joint ring 51, as described above.

Meanwhile, for the inner catheter 12, the hypotube 31 made of metal and the extending tube 32 made of resin continuing from the inner tube 21 are connected by thermally welding the extending tube 32, without using the joint ring 51. It is possible to suppress a local increase in rigidity at the middle position of the outer tube 41 in the axial line direction by applying the configuration to a configuration in which the inner opening 23b is disposed in the outer tube 41 in the initial state (the state when the relative positions of the catheters 11 and 12 are the initial positions). Further, when showing the operational effect, it may be possible to connect the extending tube 32 with the hypotube 31 by using an adhesive and it may be possible to directly connect the hypotube 31 to the inner tube 21 without using the extending tube 32.

In the configuration in which the outer tube 41 and the outer shaft 42 are connected by using the joint ring 51, the length of the joint ring 51 is set such that the marker 37 for an inner opening does not overlap the joint ring 51. Therefore, it is possible to contrast the marker 37 for an inner opening well.

The inner side rigidity reduction region 35 is formed at the inner shaft 22 such that the overlap region D is generated between the joint ring 51 and the inner shaft 22. Accordingly, it is possible to suppress the influence on an increase in rigidity due to the existence of the joint ring 51.

The present invention is not limited to that described in the embodiment, and for example, may be implemented as follows.

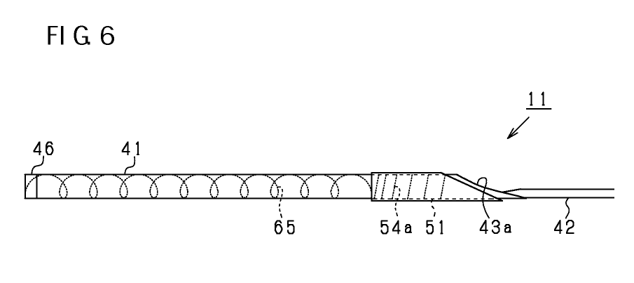

(1) In the configuration of FIG. 6 that is an applicable modified example, a coil 65 made of metal is disposed at the outer tube 41 of the outer catheter 11. The coil 65 is embedded in the base layer 44, in the same axial line as the outer tube 43, from the distal end portion of the outer tube 41 to the distal end of the joint ring 51. Accordingly, the strength of the outer tube 41 in the radial direction increases and the outer tube 41 is not easily crushed in use.

Further, it is possible not to reduce flexibility in the direction crossing the axial line direction of the outer tube 41, by making the outer diameter of the metal wire of the coil 65 smaller than that of the core material 34 or the guide wire 61 and widening the gap of the adjacent portions in the axial line direction more than the pitch of the slit 54a formed at the joint ring 51.

Further, a platinum wire maybe used for the coil 65. In this case, it is possible to know the entire length of the outer tube 41 under radiation. Even in the configuration, it is possible to prevent the marker 37 for inner opening from be covered by the coil 65 by setting the outer diameter or the gap of the metal wire of the coil 65 described above.

(2) It may be possible to perform a structure or coating that reduces locking to the inner tube 21 or another catheter, at the side opposite to the side where the outer shaft 42 is disposed, at the periphery of the outer opening 43a, that is, at the distal end side of the periphery. As the structure of reducing locking, for example, a configuration of shaping the distal end side of the periphery of the outer opening 43a such that the contact area with the inner tube 21 or another catheter becomes small, is considered.

(3) The inner opening 23b and the outer opening 43a are not limited to the configuration in which the open surfaces are inclined with respect to the axial line direction, and at least one of them may be formed to be perpendicular to the axial line direction. Further, at least one of them may be formed in a flare shape toward the proximal end side.

(4) The configuration of connecting the tube with the shaft by using the joint ring 51 may be applied to the inner catheter 12, and in this configuration, the configuration of the inner catheter 12 of the embodiment may be applied as a configuration for connecting the tube with the shaft in the outer catheter 11. In this case, it may be possible to dispose the outer opening 43a at a position further toward the distal end side than the inner opening 23b in the initial state of the catheter assembly 10. In the configuration, when a marker for contrast is separately disposed for the outer opening 43a, it is preferable to dispose the joint ring of the inner catheter 12 not to overlap the marker in the axial line direction. Further, when the configuration of the joint ring 51 is applied to the inner catheter 12, it is possible to improve the passing performance of the guide wire 61 while implementing strong connection by using the configuration according to the large width surface 53a and the inclining surface 53b.

(5) The inner side rigidity reduction region 35 or the outer side rigidity reduction region 54 is not limited to the configuration formed by the spiral slits 35a and 54a. The region 35 and 54 may be configured to be formed by a straight slit or may be configured to be formed in a mesh shape, and may be configured to be formed by reducing the thickness or the outer diameter toward the distal end side.

(6) The joint ring 51 is not limited to the continuous cylindrical shape around the axis and may have a disconnected portion at the middle position around the axis. Further, the rigidity in the radial direction reduces, but it may be possible to use a joint plate (or joint piece) made of metal instead of the joint ring 51 when suppressing a local increase in rigidity.

(7) The usage of the catheter assembly 10 is not limited to the usage for delivering another catheter such as a balloon catheter, and for example, the outer catheter 11 may be configured to be used as an aspiration catheter and the inner catheter 12 may be configured to be used as an insertion assisting tool. Further, the catheter assembly 10 may be used to deliver a self-expanding type of stent or the catheter assembly 10 may be used to open up an occlusion part or a narrowed part by using the distal end portion of the inner catheter 12 or the distal end portion of the outer catheter 11.

1. A catheter assembly comprising:
an inner member having an inner hole through which a guide wire is inserted; and
an outer member having an outer hole through which the inner member is inserted, wherein
the inner member has an inner opening, by which the inner hole is opened to the outside of the inner member, at a middle position in an axial line direction of the inner member, the inner opening is formed such that an inner open surface faces a proximal end side or inclines with respect to the axial line direction,
the outer member has an outer opening, by which the outer hole is opened to the outside of the outer member, at a middle position in an axial line direction of the outer member, and the outer opening is formed such that an outer open surface faces a proximal end side or inclines with respect to the axial line direction, on the same line as the inner opening.

2. The catheter assembly according to claim 1, wherein
the inner member has an inner tube having the inner hole and the inner opening at the proximal end portion, and an inner shaft being provided to extend toward the proximal end side from the inner tube and having a dimension that is smaller than a dimension of the inner opening in a direction perpendicular to the axial line direction, and
the outer member has an outer tube having the outer hole and the outer opening at the proximal end portion, and an outer shaft being provided to extend toward the proximal end side from the outer tube and having a dimension that is smaller than a dimension of the outer opening in a direction perpendicular to the axial line direction.

3. The catheter assembly according to claim 2, further comprising:
a contrast portion being provided at a position of the opening of the one of the inner member and the outer member, or a position adjacent thereto; and
a joint part being provided at the other one of the inner member and the outer member and having a metal region welded to a metal region of the shaft of the other one of the inner member and the outer member, and moreover connecting the shaft with the corresponding tube by welding,
wherein the joint part is provided not to overlap the contrast portion in the axial line direction.

4. The catheter assembly according to claim 2,
wherein the outer tube has a joint part being provided at a position of the outer opening or a position adjacent thereto, and having a metal region welded to a metal region of the outer shaft, and moreover connecting the outer shaft to the outer tube by welding.

5. The catheter assembly according to claim 4, wherein
the inner opening is positioned further toward a distal end side than the outer opening when relative positions of the inner member and the outer member in the axial line direction are predetermined initial positions,
the inner member has a contrast portion provided at the position of the inner opening or a position adjacent thereto, and
the length of the joint part is set not to overlap the contrast portion in the axial line direction.

6. The catheter assembly according to claim 4, wherein
the inner opening is positioned further toward a distal end side than the outer opening when the relative positions of the inner member and the outer member in the axial line direction are predetermined initial positions,
the inner shaft has a rigidity reduction structure at the position overlapping the joint part in the axial line direction when the relative positions are the initial positions, and
the rigidity reduction structure exhibits reduced rigidity of the inner shaft than that on the proximal end side.

7. The catheter assembly according to claim 4, wherein
the joint part is formed to have a cylindrical shape around the axial line and an open surface of a proximal end side thereof inclines with respect to the axial line,
the outer shaft has a tapered portion at a distal end portion thereof, the tapered portion is formed by crushing one side of the outer shaft to the other side with the axial line therebetween, and has a large width surface and an inclining surface,
the large width surface includes a portion leveled with the portion positioned further toward the proximal end side than the tapered portion throughout the axial line direction and is made wider than the portion on the proximal end side,
the inclining surface is inclined to gradually decrease the distance from the large width surface toward the distal end side,
the axial line direction of the outer shaft is the axial line direction of the outer tube as the large width surface is provided to be in contact from the inside with a portion, the portion being the inner circumferential surface of a tube wall defining the opening and the periphery of the proximal end side of the joint part and being in the same straight line of the proximal end portion of the opening, and
the joint part and the outer shaft are welded at the contact portion.

8. The catheter assembly according to claim 5, wherein
the inner opening is positioned further toward a distal end side than the outer opening when the relative positions of the inner member and the outer member in the axial line direction are predetermined initial positions,
the inner shaft has a rigidity reduction structure at the position overlapping the joint part in the axial line direction when the relative positions are the initial positions, and
the rigidity reduction structure exhibits reduced rigidity of the inner shaft than that on the proximal end side.

9. The catheter assembly according to claim 5, wherein
the joint part is formed to have a cylindrical shape around the axial line and an open surface of a proximal end side thereof inclines with respect to the axial line,
the outer shaft has a tapered portion at a distal end portion thereof,
the tapered portion is formed by crushing one side of the outer shaft to the other side with the axial line therebetween, and has a large width surface and an inclining surface,
the large width surface includes a portion leveled with the portion positioned further toward the proximal end side than the tapered portion throughout the axial line direction and is made wider than the portion on the proximal end side,
the inclining surface is inclined to gradually decrease the distance from the large width surface toward the distal end side,
the axial line direction of the outer shaft is the axial line direction of the outer tube as the large width surface is provided to be in contact from the inside with a portion, the portion being the inner circumferential surface of a tube wall defining the opening and the periphery of the proximal end side of the joint part and being in the same straight line of the proximal end portion of the opening, and
the joint part and the outer shaft are welded at the contact portion.

10. The catheter assembly according to claim 6, wherein
the joint part is formed to have a cylindrical shape around the axial line and an open surface of a proximal end side thereof inclines with respect to the axial line,
the outer shaft has a tapered portion at a distal end portion thereof,
the tapered portion is formed by crushing one side of the outer shaft to the other side with the axial line therebetween, and has a large width surface and an inclining surface,
the large width surface includes a portion leveled with the portion positioned further toward the proximal end side than the tapered portion throughout the axial line direction and is made wider than the portion on the proximal end side,
the inclining surface is inclined to gradually decrease the distance from the large width surface toward the distal end side,
the axial line direction of the outer shaft is the axial line direction of the outer tube as the large width surface is provided to be in contact from the inside with a portion, the portion being the inner circumferential surface of a tube wall defining the opening and the periphery of the proximal end side of the joint part and being in the same straight line of the proximal end portion of the opening, and
the joint part and the outer shaft are welded at the contact portion.

11. The catheter assembly according to claim 8, wherein
the joint part is formed to have a cylindrical shape around the axial line and an open surface of a proximal end side thereof inclines with respect to the axial line,
the outer shaft has a tapered portion at a distal end portion thereof,
the tapered portion is formed by crushing one side of the outer shaft to the other side with the axial line therebetween, and has a large width surface and an inclining surface,
the large width surface includes a portion leveled with the portion positioned further toward the proximal end side than the tapered portion throughout the axial line direction and is made wider than the portion on the proximal end side,
the inclining surface is inclined to gradually decrease the distance from the large width surface toward the distal end side,
the axial line direction of the outer shaft is the axial line direction of the outer tube as the large width surface is provided to be in contact from the inside with a portion, the portion being the inner circumferential surface of a tube wall defining the opening and the periphery of the proximal end side of the joint part and being in the same straight line of the proximal end portion of the opening, and
the joint part and the outer shaft are welded at the contact portion.
